(19) United States
(12) Patent Application Publication
(10) Pub. No.: US 2016/0361398 A1
(43) Pub. Date: Dec. 15, 2016

(54) METHOD FOR THE TREATMENT OF MULTIPLE SCLEROSIS

(71) Applicant: Vereniging voor Christelijk Wetenschappe Onderwijs, Amsterdam (NL)

(72) Inventors: Yvette Van Kooyk, Amsterdam (NL); Wendy Wilhelmina Josephina Unger, Amsterdam (NL)

(21) Appl. No.: 14/736,130

(22) Filed: Jun. 10, 2015

(51) Int. Cl. A61K 39/00

(52) U.S. Cl. CPC A61K 39/0007

(57) ABSTRACT

The disclosure is in the field of immunology and provides means and methods for the treatment of multiple sclerosis. More in particular, the disclosure relates to means and methods for inducing immune tolerance to an antigen, wherein the antigen is covalently attached to a sialylated oligosaccharide, in particular, 6′-sialyl-N-acetyllactosamine. More in particular, the disclosure provides means and methods for inducing immune tolerance against an antigen derived from myelin, such as a myelin component, such as myelin oligodendrocyte glycoprotein, a well-known target in experimental autoimmune encephalomyelitis, a murine multiple sclerosis model.

### TECHNICAL FIELD

The disclosure is in the field of immunology and provides means and methods for the treatment of multiple sclerosis. More in particular, the disclosure relates to means and methods for inducing immune tolerance to an antigen, wherein the antigen is covalently attached to a sialylated oligosaccharide, in particular, 6′-sialyl-N-acetyllactosamine. More in particular, the disclosure provides means and methods for inducing immune tolerance against an antigen derived from myelin, such as a myelin component, such as myelin oligodendrocyte glycoprotein, a well-known target in experimental autoimmune encephalomyelitis, a murine multiple sclerosis model.

### BACKGROUND

Multiple sclerosis (MS) is a chronic autoimmune disease of the central nervous system (CNS) that mainly affects young adults, and is generally diagnosed between the ages of 20 and 40 years.^{[1]} Experimental autoimmune encephalomyelitis (EAE), induced by immunization of susceptible mouse strains with myelin oligodendrocyte glycoprotein peptides (MOG 35-55) or other myelin components, provides a useful animal model for human multiple sclerosis.

Self-reactive encephalitogenic CD4-positive (also called CD4⁺) T cells are critically involved in the initiation and development of EAE. In addition to the role of self-reactive effector CD4-positive T cells in the progression of EAE, other cell types such as macrophages, resident microglia and astrocytes also contribute to immuno-pathogenesis in MS/EAE. When activated, these cells can act as antigen-presenting cells (APC), producing pro-inflammatory cytokines and chemokines in the local environment.^{[2,3]}

Thus far, there are no effective therapeutics that can stop the progression of MS or significantly restore neurological function.^{[7]} Soluble peptides representing encephalitogenic epitopes administered orally and via other routes have proved effective in suppressing EAE in mouse models during the induction phase of EAE;^{[4-6]} however, the effectiveness of this strategy was elusive in clinical trials involving MS patients.^{[7]}

Several mechanisms have been proposed to explain the phenomenon of such immune tolerance. Evidence from animal models suggests that immune tolerance is associated with anergy and deletion of self-reactive T cells and generation of regulatory T cells, which can be induced by partial or complete blockade of co-stimulatory signaling.^{[8-10]} In addition, immunomodulatory cytokines TGF-beta and IL-10 are strongly associated with regulatory T cells and immune tolerance.^{[9]} The transcription factor Foxp3 is critical to CD41 CD251 regulatory T-cell differentiation, probably through inhibition of NF-kB activation and nuclear translocation.^{[11]} Thus, T-cell anergy, deletion, generation of regulatory T cells and production of anti-inflammatory cytokines mediate immune tolerance in physiological and pathological conditions.

As CD4⁺ T cells are key contributors to the underlying pathogenic mechanisms responsible for the onset and progression of most autoimmune diseases, they are the prime target for therapeutic strategies. One method for restoring self-tolerance is to exploit the endogenous regulatory mechanisms that govern CD4⁺ T-cell activation. Turley and Miller^{[12]} discuss tolerance strategies with the common goal of inducing antigen (Ag)-specific tolerance, which focus on the use of peptide-specific tolerance strategies.

One technique for restoring self-tolerance is to exploit the endogenous regulatory mechanisms that govern CD4⁺ T-cell activation. Typically, endogenous ligation of the T-cell receptor (TCR) by peptide/MHC class II alone produces a signal of insufficient strength to activate a CD4+ T cell and can instead induce functional anergy or deletion. As a consequence, additional APC-derived co-stimulatory signals (e.g., CD80/86 engagement of CD28) are required to lower the threshold required for successful T-cell activation.

This “two-signal” hypothesis predicts that TCR stimulation in the absence of costimulatory signals leads to CD4⁺ T-cell anergy, tolerance, and/ or depletion.^{[13]} Therefore, either TCR ligation in the absence of costimulatory signals or exogenous targeting of the costimulatory pathway would appear to be a logical target of therapeutic strategies to down-regulate the pathologic functions of autoreactive CD4+ T cells. In light of this, various therapeutic approaches have been designed to block autoreactive CD4+ T-cell function during autoimmune disease, including the administration of blocking antibodies directed against a variety of epitopes including CD3, CD4, CD28, CD40, CD80, CD86, CD154, ICOS, OX40, and 4-1BB, as well as CTLA4-Ig.^{[14, 15]}

However, these treatment strategies, if administered over a long time period, often result in either non-specific immune suppression or other undesirable side effects.

Turley and Miller^{[12]} also discuss techniques with the common purpose of inducing Ag-specific tolerance by specifically targeting the TCR to avoid detrimental influences on non-specific/bystander immune processes. The direct targeting of autoreactive T cells is a promising treatment strategy for autoimmune disease, resulting in Ag-specific unresponsiveness without global immunosuppression.

There are currently four different protocols employed for inducing peptide-specific immune tolerance: altered peptide ligand (APL)-induced tolerance, mucosal (oral-nasal)-induced tolerance, soluble-peptide-induced tolerance, and ECDI-coupled-cell-induced tolerance.^{[12]}

Moreover, Bar-Or et al.^{[16]} describe the induction of antigen-specific tolerance in multiple sclerosis after immunization with DNA encoding myelin basic protein. They investigated the immune modulation by BHT-3009, a tolerizing DNA vaccine encoding full-length human myelin basic protein, in patients with multiple sclerosis (MS). BHT-3009 was found to be safe and well tolerated, provided favorable trends on brain MRI, and produced beneficial antigen-specific immune changes. These immune changes consisted of a marked decrease in proliferation of interferon-gamma (IFN-gamma)-producing, myelin-reactive CD4+ T cells from peripheral blood and a reduction in titers of antigen (myelin)-specific autoantibodies from cerebral spinal fluid. Moreover, a concordant reduction of inflammatory lesions on brain MRI was observed.

Jiang Z. et al.^{[17]} describe how intravenous administration of MOG(35-55) peptide suppresses experimental autoimmune encephalomyelitis. They induced tolerance by intravenous administration of MOG(35-55) peptide and determined the effect of this approach on intracellular signaling pathways of the IL-23/IL-17 system, which is essential for the pathogenesis of MS/EAE.

Despite the availability of many promising leads for the treatment of multiple sclerosis, there is still a need for improved means and methods that increase the tolerance of the MS patient for antigens of the central nervous system (CNS self-antigens), in particular, myelin-derived antigens like MOG.

### BRIEF SUMMARY

The disclosure provides a compound comprising a sialylated oligosaccharide covalently attached to an antigen, for use in the treatment of multiple sclerosis, wherein the antigen is derived from myelin and wherein the sialylated oligosaccharide is 6′-sialyl-N-acetyllactosamine, also known as Neu5Ac(α)2-6Gal(β)1-4GlcNAc or 6′-SLN.

### BRIEF DESCRIPTION OF THE DRAWINGS

FIGS. 1A through 1G: Treg induction and dampening of autoreactive T-cell activity by DCs targeted with α-2-6-Sia-MOG35-55.

FIGS. 1A and 1B: Naïve MOG35-55-specific CD4 (2D2) T cells were co-cultured with Sia-MOG or non-sialylated MOG-loaded DCs. Proportion of Foxp3+ regulatory T cells (Treg) and IFN-γ+ effector T cells (T eff) 2D2 cells was analyzed.

FIG. 1C: The amount of secreted IFN-γ was measured.

FIG. 1D: (Left panel) Expression of T-bet, which is a measure for IFN-γ-producing effector CD4 T cells (Th1). (Right panel) Foxp3 mRNA, which is a measure for Treg cells that suppress, were determined using qRT-PCR and subsequently normalized to housekeeping gene GAPDH, which is equal under all conditions.

FIG. 1E: Cytokine secretion of MOG-specific Th1 (IFN-gamma) and Th17 effector cells (IL-17) is measured.

FIGS. 1F and 1G: The spleens of mice suffering from experimental autoimmune encephalomyelitis (EAE) were re-stimulated in vitro with DCs loaded either with α-2-6-Sia-MOG35-55 (black bars) or native, non-sialylated MOG35-55 (grey bars). As a control, PBS-treated DCs (white bars) and the spleens of naive mice were assessed. On day 4 (FIG. 1E), MOG-specific T-cell proliferation following [3H] thymidine incorporation and amounts of IFN-γ (FIG. 1F) in culture supernatants were determined. Data are the means of triplicate cultures from three animals per condition. Values represent the means±SEM of triplicate cultures.

FIG. 2: A graph depicting the negative control experiment with OVA-loaded dendritic cells challenged with OVA that did not induce tolerogenic cells above background levels in a population of OT-II cells.

### DETAILED DESCRIPTION

It was found that such sialylated antigens could be exploited to prevent or dampen inflammation in an antigen-specific manner.

The term “sialylated antigen” is used herein to refer to an antigen that is covalently attached to a sialylated oligosaccharide, in particular, Neu5Ac(α)2-6Gal(β)1-4GlcNAc.

Sialic acid is a generic term for the N- or O-substituted derivatives of neuraminic acid, a monosaccharide with a nine-carbon backbone. It is also the name for the most common member of this group, N-acetylneuraminic acid (Neu5Ac or NANA). Sialic acids are found widely distributed in animal tissues and to a lesser extent in other organisms, ranging from plants and fungi to yeasts and bacteria, mostly in glycoproteins and gangliosides (they occur at the end of sugar chains connected to the surfaces of cells and soluble proteins).

In humans, the brain has the highest sialic acid concentration where they have an important role in neural transmission and ganglioside structure in synaptogenesis. In general, the amino group bears either an acetyl or a glycolyl group, but other modifications have been described. These modifications, along with linkages, have shown to be tissue-specific and developmentally regulated expressions, so some of them are only found on certain types of glycoconjugates in specific cells.

Sialic acids are the outermost monosaccharides on glycan chains of glycoproteins and glycolipids, attached to the underlying glycans with α2-3-, α2-6- or α2-8-linkage^{[19]} and, as such, the recognition elements for selectins and Sia-binding Ig-like lectins (siglecs).^{[19,20]}

As a proof-of-principle, a well-accepted model system was employed for multiple sclerosis wherein a fragment (amino acid 35 -55) of the antigen myelin oligodendrocyte glycoprotein (MOG 35-55, SEQ ID NO:1) was sialylated by covalently attaching a sialylated oligosaccharide, in particular 6′-Sialyl-N-acetyllactosamine (6′-SLN or Neu5Ac(α)2-6Gal(β)1-4GlcNAc). This sialylated oligosaccharide is commercially available as SLN306 from Dextra Laboratories Ltd., Reading UK.

MOG is one of the well-known targets of autoreactive T cells in experimental autoimmune encephalomyelitis (EAE), a murine multiple sclerosis model.^{[18]} In the examples section, the preparation of an α-2,6-Sia MOG 35-55 peptide (also referred to herein as α-2-6-sia-MOG) is shown.

The term “derived from myelin” as used herein refers to compositions and molecules that may be derived from myelin, either in the manufacturing sense of the word “derived” or in the theoretical sense. A peptide fragment that is synthetically manufactured on the basis of a given sequence of a myelin molecule is considered herein as being “derived” from myelin. In other words, an antigen is derived from myelin if myelin is the source, either in a practical sense, such as a purification process, or in an intellectual sense, for instance, when a sequence of a particular fragment is selected and subsequently manufactured. In yet other terms, “derived” means to obtain a chemical substance actually or theoretically from a parent substance.

Well-known antigens that may be derived from CNS antigens are, in particular, antigens that may be derived from myelin. Such antigens are, for instance, Myelin Oligodendrocyte Glycoprotein (MOG), Myelin Basic Protein (MBP), Myelin-Associated Glycoprotein (MAG), spinal cord homogenate (SCH), purified myelin, myelin Proteolipid Protein (PLP) or peptides of these proteins.

Without wanting to be bound by theory, the hypothesis was made that pro-inflammatory, myelin-reactive CD4+ T-cells, in particular, pathogenic (IL-17 producing) Th17 and (IFN-γ-producing) Th1 cells are involved in disease pathogenesis.^{[2]} These T-cells can be directed against myelin-containing antigens such as myelin oligodendrocyte glycoprotein (MOG)^{[8]} or myelin basic protein (MBP)^{[9]} or proteolipid protein (PLP) (Reeks et al., Clinical Immunology 2015, S1521-6616), which are candidate auto-antigens in MS, as enhanced levels of IFN-γ producing MOG-specific T-cell and MBP-specific T-cells have been found to be important in MS.

It is demonstrated herein that the sialylation of an antigen, notably a peptide, changes the program of antigen-presenting cells (APCs) such as dendritic cells (DCs) toward a tolerogenic, suppressive stage.

For that purpose, DC were pulsed with α-2-6-Sia-MOG35-55 and a non-sialylated MOG 35-55 peptide as a control, and cultured for six days with MOG-responsive CD4⁺ T cells (2D2 T cells (Bettelli et al., J. Exp. Med., 2003).

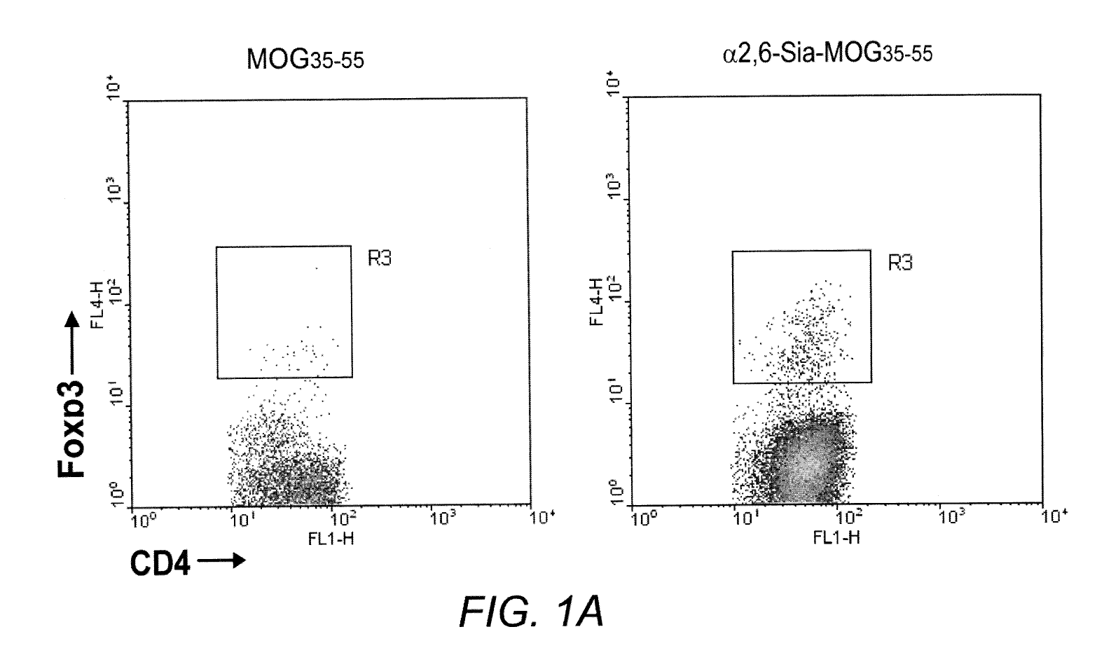

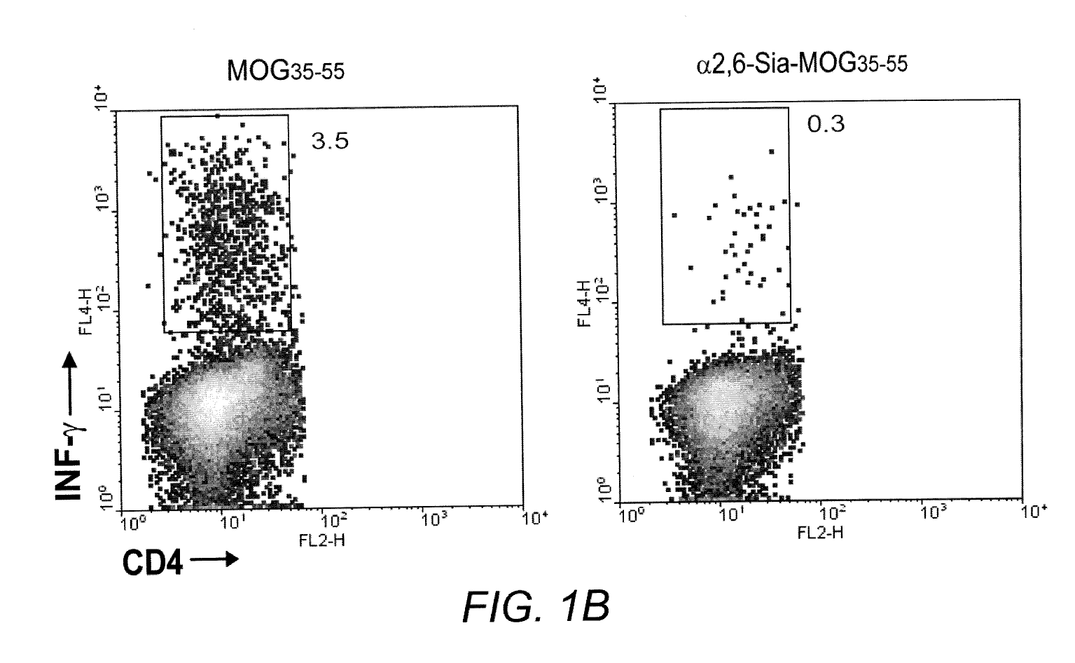

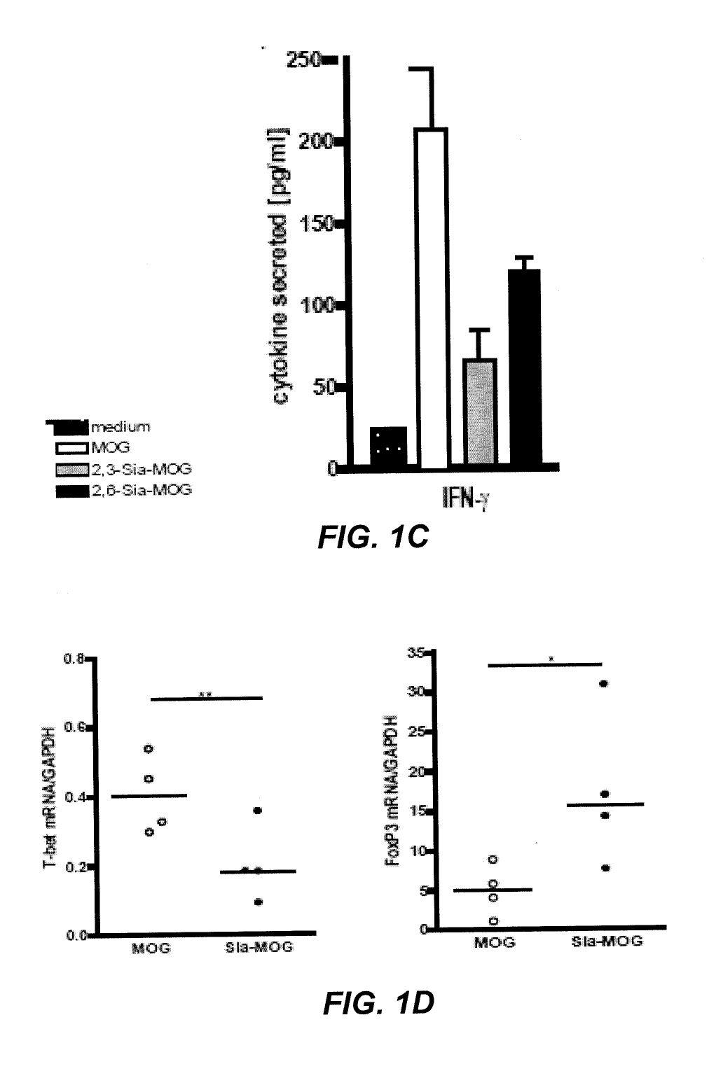

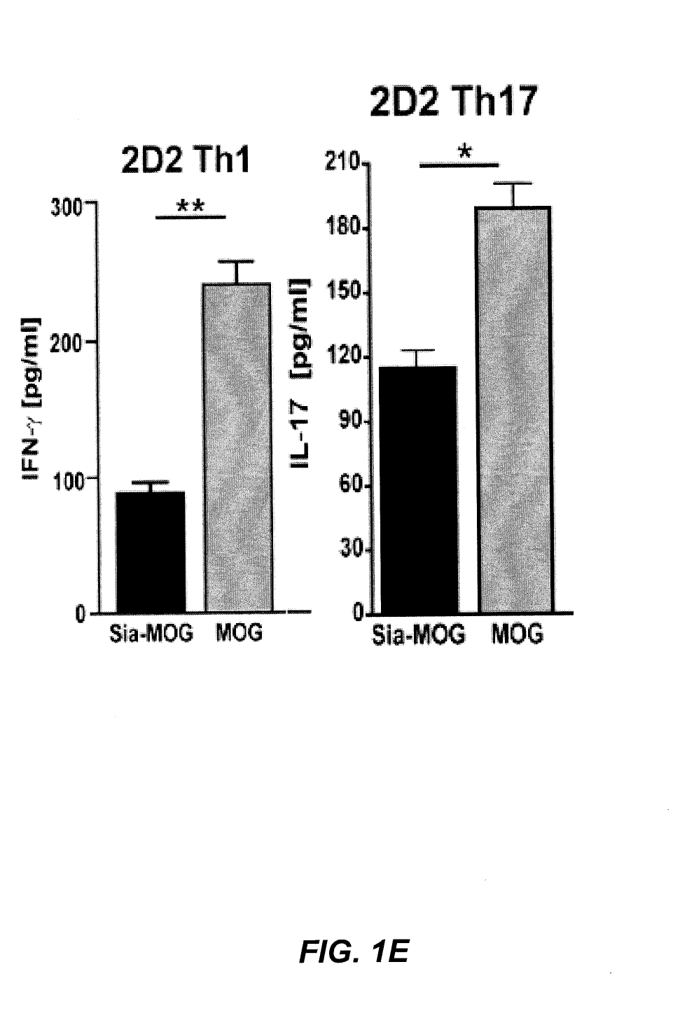

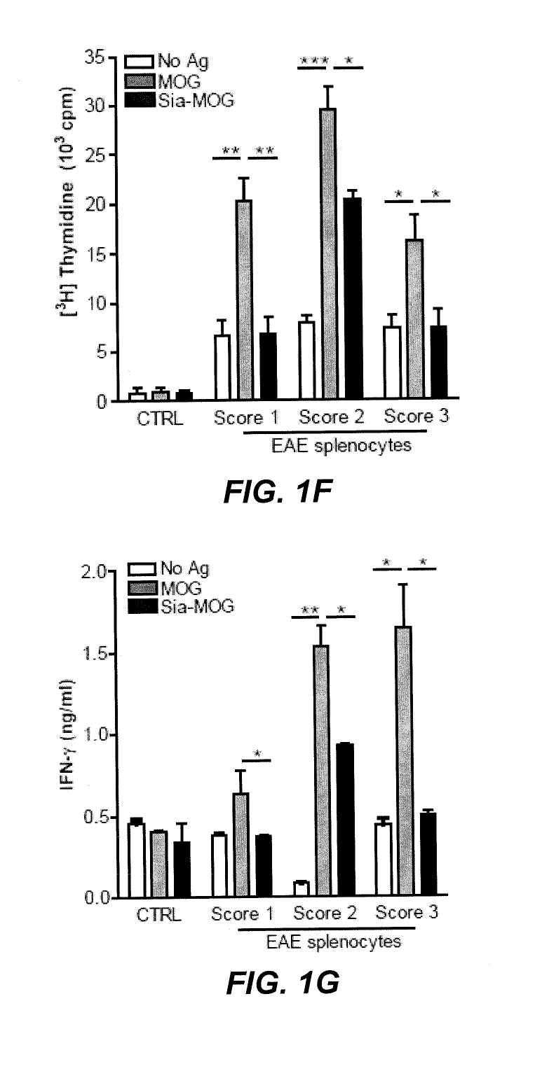

It was observed that DCs exposed to non-sialylated MOG peptide stimulated MOG-specific CD4 T cells, as these T cells start to produce significant amounts of IFN-γ (FIG. 1B, left panel (CD4+ IFN-γ+ (3.5%).

When DC were exposed to the α-2-6-Sia-MOG35-55, no MOG effector T cells were induced (only 0.3%).

However, the majority of the CD4 T cells co-express Foxp3, which is a hallmark for immune suppressive T cells (FIG. 1A, right panel). These suppressive Foxp3+CD4+ T cells were not induced when DC were exposed to the non-sialylated MOG35-55 peptide. This clearly demonstrates that the α-2-6-Sialylation of the MOG peptides instructs DC to modify CD4+ T cell responses and deviate from becoming IFN-γ-producing MOG-specific effector T cells (this is illustrated in FIGS. 1A and 1B).

Analysis of the culture supernatants of the DCs exposed to non-sialylated MOG35-55 or α-2-6-Sia-MOG that were co-cultured with MOG-specific CD4⁺ (2D2) T-cells confirmed that only T cells primed by non-sialylated MOG produced significant amounts of IFN-γ (FIG. 1C), which is the effector cytokine.

Notably, CD4⁺ T cells primed by α-2-6-Sia-MOG35-55-loaded DCs showed a reduction of this effector T-cell cytokine.

Furthermore, qRT-PCR analysis of MOG-specific CD4+ T cells, isolated on day 6 of co-culture with DCs pulsed with unsialylated (native) MOG- or α-2-6-Sia-MOG, confirmed the data that higher mRNA levels of the effector cell marker Th1 lineage-specific transcription factor T-bet, were detected in T cells activated by DCs pulsed with non-sialylated MOG35-55 than in T cells primed by DCs loaded with α-2-6-Sia-MOG35-55 (FIG. 1D, left panel).

Instead, DCs exposed to α-2-6-Sia-MOG35-55 instructed the T cells to express high levels of Foxp3, the marker for suppressive activity of the CD4T cells (FIG. 1D, right panel). This expression is associated with and indispensable for suppressive function of regulatory T cells (Gavin et al., Nature 445(7129); 771-5 Epub 2007). In contrast, T cells activated by DCs pulsed with non-sialylated MOG35-55, hardly expressed Foxp3 mRNA (FIG. 1D, right panel).

This finding shows that sialylation of antigens (MOG) may also be exploited to dampen existing effector cells. As stated hereinbefore, Th1 and Th17 are the effector CD4T cells that cause inflammation in MS. Therefore, Th1 (FIG. 1E, left panel) and Th17 cells (FIG. 1E, right panel) were generated from 2D2 CD4 T cells in vitro and observed that their secretion of effector cytokines was significantly reduced when encountering α-2,6-Sia-MOG35-55-loaded DCs. This was not observed when these effector cells were co-cultured with DCs loaded with non-sialylated MOG 35-55 (FIG. 1E). Thus, it was concluded that T cells generated in the presence of DCs exposed to α-2,6-Sia-MOG35-55, indeed were functional in that they were able to suppress MOG-specific effector T cells and, therefore, able to dampen ongoing inflammation. It follows that the disclosure may be applied for treating ongoing inflammation, such as the inflammation observed in MS patients, when, at the peak of the disease, auto-reactive, myelin-specific CD4⁺ and CD8⁺ T cells are involved in the demyelination of the neurons.

To examine this even further, testing was performed to determine whether DCs exposed to α-2,6-Sia-MOG35-55 were able to inhibit MOG-specific effector T cells that were obtained from splenocytes from mice suffering from experimental autoimmune encephalomyelitis (EAE). To this effect, CD4 effector T cells were harvested from splenocytes of mice at various EAE clinical scores, representing different stages of effector T cell number and activity, wherein score 1 is the onset of disease and score 2 and 3 are the more severe phases of the disease.

Testing was performed to determine whether DCs ex vivo loaded with Sia-MOG could inhibit the proliferation of the MOG-specific effector CD4 T cells that were obtained from EAE mice.

DCs loaded with α-2-6-Sia-MOG35-55 or non-sialylated MOG35-55 were cultured with EAE splenocytes and, after 4 days of culture, [3H]thymidine incorporation was determined as a measure of proliferation and division of CD4 T cells (FIG. 1 F). It was observed that non-sialylated MOG-loaded DC stimulate the proliferation and division of MOG-specific CD4 T cells obtained from EAE splenocytes of mice with different scores. It was also observed that DCs loaded with α-2-6-Sia-MOG35-55 showed a clear reduction of proliferation, indicating that T cells suppress the activation of MOG-reactive effector cells present in the onset (score 1) but also at later stage of the disease (score 2 and 3, FIG. 1F).

To determine the functionality of the MOG-reactive effector cells, IFN-γ production was measured (FIG. 1G).

It was observed that DCs loaded with non-sialylated MOG peptide enhance the ex-vivo MOG-reactive effector T cells in EAE mice with different scoring. Also in this case, DCs loaded with α-2-6-Sia-MOG35-55 suppressed the production of IFN-γ, indicating that α-2-6-Sia-MOG35-55 instructs DC with an immune suppressive programming that induces the differentiation of FoxP3 MOG-specific suppressive T cells that are functionally active and are able to inhibit inflammatory MOG-specific T cells that cause EAE or MS.

As total splenocytes were incubated with the α-2-6-Sia-MOG35-55-loaded DCs for four days, it is likely that the observed suppression was not mediated via de novo-induced CD4⁺ Tregs. However, DCs loaded with α-2-6-Sia-MOG35-55 promoted the induction of Foxp3 mRNA and protein expression in naïve, non-sialylated MOG35-55-responsive 2D2 T cells^{[22]} (FIGS. 1C and 1E). In contrast, non-sialylated, native MOG35-55-loaded DCs induced the differentiation of 2D2 T cells into Th1, as indicated by IFN-γ production and T-bet mRNA expression (FIGS. 1C and 1D).

Notably, DCs only gained tolerogenic properties when a sialylated oligosaccharide was covalently coupled to the antigen and not when the sialylated oligosaccharide and the antigen were provided separately.

These data show that covalent coupling of a sialylated oligosaccharide to an antigen, wherein the sialylated oligosaccharide preferably comprises Neu5Ac(α)2-6Gal(β)1-4GlcNAc, instruct DCs in an antigen-specific tolerogenic programming, enhancing Treg and reducing inflammatory T cells. It was also demonstrated that providing DCs with a thus sialylated antigen has a potent dual tolerogenic function: strongly promoting de novo Treg induction while simultaneously inhibiting effector T-cell differentiation, even under inflammatory conditions.

The data show that, in particular, DCs instigate the switch in T-cell quality as was demonstrated that DCs become tolerogenic upon uptake of soluble sialylated antigens. Thus, sialylated glycans drastically alter DC and T cell responses and provide a novel target for modulation in a broad range of immunopathologies.

The data also show that the effect is antigen-specific. When ovalbumin (OVA) was used as a control antigen, the immune response against OVA remained completely intact, whereas the immune response against the antigen (such as MOG), covalently coupled to a sialylated oligosaccharide was suppressed.

Vice versa, when OVA was coupled to a sialylated oligosaccharide such as 6′-Sialyl-N-acetyllactosamine (6′-SLN or Neu5Ac(α)2-6Gal(β)1-4GlcNAc), the same phenomenon was observed, namely, DCs that were exposed to α-2-6-Sia-OVA were capable of inducing immune tolerance against OVA while leaving the immune reactivity against other antigens completely intact. In addition, it was also here observed that α-2-6-Sia-OVA induced only tolerance against OVA (see above) and not to MOG.

The data presented here should not be interpreted as narrow as that only a MOG peptide may be used to induce immune tolerance. Identical effects were observed with completely unrelated antigens, such as CNS antigens, such as myelin, Myelin Oligodendrocyte Glycoprotein (MOG), Myelin Basic Protein (MBP), spinal cord homogenate (SCH), Myelin-Associated Glycoprotein (MAG), purified myelin, myelin Proteolipid Protein (PLP) and peptides of these proteins.

### EXAMPLES

### Example 1

### Modification of Antigens with Sialylated Glycans

To obtain sialylated MOG (Sia-MOG), maleimide-activated 6′-sialyl-N-acetyllactosamine (SLN306; Neu5Ac(α)2-6Gal(β)1-4GlcNAc; DEXTRA Labs, UK) and 3′-sialyl-N-acetyllactosamine (SLN302; Neu5Acα2-3Galβ1-4Glc) were conjugated to thio-modified MOG35-55 peptide through thiol-ene reactions. The glycans were activated with the bi-functional cross-linker 4-N-maleimidophenyl butyric acid hydrazide (MPBH, Pierce, USA). The MOG35-55 sequence was modified with an added cysteine at the N-terminus to allow for the coupling with the activated glycans.

The hydrazide moiety of MPBH was covalently linked to the reducing end of the carbohydrate via reductive amination at a 3:1 molar ratio. Briefly, a mixture of MPBH (3 eq.), 3′-sialyl-N-acetyllactosamine (or 6′-sialyl-N-acetyllactosamine) (1 eq.) and picoline borane (10 eq., Sigma-Aldrich, Germany) dissolved in DMSO/AcOH (8:2) and 1% TFA was incubated for 2 hours at 65° C. After cooling down to room temperature, 4 volumes of dichloromethane (Biosolve) were added and the mixture was vortexed thoroughly. Subsequently, 10 volumes of diethyl ether (Biosolve) were added and stirred briefly until glycan-MPBH had completely precipitated. MPBH-glycans were pelleted by centrifugation (2 minutes at 14,000 rpm ˜20,000 g) and washed twice with diethyl ether. The obtained glycan-MPBH pellet was resuspended in aqueous buffer with 0.1% TFA (trifluoroacetic acid, Sigma) and lyophilized, followed by purification over a 22×250 mm Vydac MS214 prep C18 column (Grace Alltech, elution water/acetonitrile, gradient 3% to 50% of acetonitrile in 40 minutes) on a Dionex prep 3000 HPLC system. The fractions containing the glycan-MPBH were pooled and lyophilized. The derivatization and purity of the MPBH-glycans was confirmed by HPLC (Vydac 218MS C18 5 μm 4.6×250 mm, Grace Alltech) and MS spectrometry (LCQ-Deca XP lontrap Thermo Finnigan mass spectrometer in positive mode using nanospray capillary needle).

The primary sequence of human MOG 35-55 is: Met-Glu-Val-Gly-Trp-Tyr-Arg-Pro-Pro-Phe-Ser-Arg-Val-Val-His-Leu-Tyr-Arg-Asn-Gly-Lys (SEQ ID NO:1). A cysteine residue was attached to the N-terminus. Cysteine-modified MOG35-55 peptide was produced by solid phase peptide synthesis using Fmoc chemistry with a Symphony peptide synthesizer (Protein Technologies Inc., USA). When the experiments were repeated with mouse MOG (Met-Glu-Val-Gly-Trp-Tyr-Arg-Ser-Pro-Phe-Ser-Arg-Val-Val-His-Leu-Tyr-Arg-Asn-Gly-Lys (SEQ ID NO:2)), the results were identical.

The final reaction of MOG35-55SH with the MPBH-carbohydrates was performed at a molar ratio of 1:1.5. Briefly, the peptide (1 eq.) was dissolved in DMSO and added to the carbohydrate-MPBH (1.5 eq.) with 50 mM TEA. After 2 hours of reaction at room temperature, the glycated peptide was purified by HPLC using a Vydac MS214 prep C18 column 10×250 mm (Grace Alltech, elution water/acetonitrile, gradient 10% to 50% of acetonitrile in 40 minutes). The fractions containing the glycopeptide were pooled and lyophilized. The derivatization and purity of the glycated peptide were confirmed by HPLC (Vydac 218MS C18 5 μm 4.6×250 mm, Grace Alltech) and ESI-MS spectrometry (LCQ-Deca XP Iontrap Thermo Finnigan mass spectrometer in positive mode using nanospray capillary needle).

### Example 2

### T Helper Differentiation and Testing of Suppressive Capacity

Antigen-specific nave CD4+CD62LhiCD25− T cells were purified from spleen and LN cell suspensions obtained from 2D2 mice using the Dynal mouse CD4+ CD62L+ T cell isolation kit II mouse (Miltenyi Biotec, Bergisch Gladbach, Germany) or by sorting on a MoFlo (DakoCytomation, Glostrup, Denmark). The resulting naïve CD4+ CD62L+CD25− T cell populations were typically 95% pure as assessed by flow cytometry. Naïve CD4+ T cells (5×10⁴) were added to wells containing DCs (1×10⁴) that were pulsed with indicated concentrations of antigen modified with a sialylated oligosaccharide or native antigen 3 hours prior. After 2 days, 10 U/ml recombinant mouse IL-2 (Invitrogen, Bleijswijk, The Netherlands) was added. T-cell polarization was evaluated on day 6 by intra-cellular staining for Foxp3 and IFN-γ following 5 hours restimulation with phorbol 12-myristate 13-acetate (PMA; 30 μg/ml)/ionomycin (Sigma; 500 ng/ml) in the presence of Brefeldin A (Sigma; 5 μg/ml). For inhibition of effector Th1 and Th17 cells, Naïve 2D2 T cells were converted in Th1 or Th17 cells by culturing; one week later, effector T cells re-stimulated with DC pulsed with MOG or Sialylated MOG. After 24 hours, the supernatant was harvested for presence of IFN-γ or IL 17 as measure of activity of Th1 and Th17 effector cells, respectively.

### Example 3

### Suppressive Capacity of DCs Exposed to α-2-6-Sia-MOG35-55 to Inhibit Effector T Cells Obtained from Mice that Suffered Experimental Autoimmune Encephalomyelitis

Splenocytes from mice that suffered EAE were re-stimulated ex-vivo with DCs loaded with either non-sialylated MOG or α-2-6-Sia-MOG35-55 and as a control, DC with no antigen. DC and T cells were cultured as described in Example 2, as described above, for 4 days. 3H and IFN-γ was determined as described above.

### Example 4

### Cytokine Analysis

Cytokine production by T cells and DCs was assessed by cytometric bead arrays using the CBA Th1/2/17 kit and mouse inflammation kit, respectively (BD Biosciences), or by ELISA using specific antibody pairs (eBiosciences) following the manufacturers' instructions.

### Example 5

### Mice

C57BL/6 mice were purchased from Charles River Laboratories (Maastricht, The Netherlands) and used at 8-12 weeks of age. 2D2 TCR transgenic mice were bred and housed in the animal facilities of VUmc, Erasmus MC, and the TWINCORE Institute under specific pathogen-free conditions. All experiments were approved by the Animal Experiments Committee of the Erasmus MC and VUmc and performed in accordance with national and international guidelines and regulations.

### Example 6

### Dendritic Cells

Splenic DCs: Spleens isolated from C57BL/6 mice were incubated in HE medium (RPMI1640 media containing 10% FCS, 10 mM EDTA, 20 mM HEPES, 50 U/ml penicillin, 50 μg/ml streptomycin) supplemented with 1 WU/ml Liberase-TL (Roche Diagnostics GmbH, Manheim, Germany) and 50 μg/ml DNase I (Roche) for 30 minutes at 37° C. or until digested. Red blood cells were lysed using ACK lysis buffer and undigested material was removed by filtration. DCs were purified by positive selection using anti-CD11c MACS microbeads (Miltenyi Biotec, Bergisch Gladbach, Germany) according to the manufacturer's protocol. The resulting CD11c+MHC-II+ populations were typically 94% pure as assessed by flow cytometry.

Bone marrow-derived DCs: BMDCs were cultured as described by Lutz et al. with minor modifications as described earlier (Singh et al., Mol. Immunol. 47:164, 2009).

### Example 7

### Antibodies

The FITC-labeled antibodies used were anti-CD11c (N418), anti-PDL2 (122), anti-CD25 (PC61.5); the PE-labeled antibodies were anti-CD8b (H35-17.2), anti-CD4 (GK1.5), anti-CD80 (16-10A1), anti-CD86 (GL1), anti-CD40 (1C10), anti-MHC class II (MS/ 114.15.2), anti-PDL1 (MIH5), anti-Vβ5 (MR9-4); the APC-labeled antibodies were anti-CD62L (MEL-14), anti-FoxP3 (FJK-169), and anti-IFN-γ (XMG1.2). Biotin-labeled anti-Vα2 TCR (B20.1) was detected using ALEXA FLUOR® 488-labeled streptavidin (Jackson Immunoresearch, West Grove, Pa., USA). Anti-CD86, -MHC-II, -Vα2, -Vβ5, -CD62L and -IFN-γ antibodies were purchased from BD Pharmingen (BD Biosciences, San Jose, Calif., USA); all other antibodies were obtained from eBiosciences (eBiosciences, San Diego, Calif., USA).

### Example 8

### Cytokine Analysis and Antibodies

Cytokine production by T cells and DCs was assessed using the mouse CBA Th1/2/17 kit and inflammation kit, respectively (BD Biosciences). The FITC-labeled antibodies used were anti-CD11c (N418), anti-PDL2 (122), anti-CD25 (PC61.5) -CD4 (RM4-5),-CD8b (eBIOH35); PE-labeled antibodies used: anti-CD8b (H35-17.2), anti-CD4 (GK1.5), anti-CD80 (16-10A 1), anti-CD86 (GL1), anti-CD40 (1C10), anti-MHC class II (MS/114.15.2), anti-PDL1 (MIH5); APC-labeled antibodies used: anti-CD62L (MEL-14), anti-Foxp3 (FJK-169), and anti-IFN-γ (XMG1.2). Anti-CD86, -MHC-II, -CD62L and -IFN-γ antibodies were purchased from BD Pharmingen (BD); all other antibodies were obtained from eBiosciences (eBiosciences, San Diego, Calif., USA).

### Example 9

### Statistical Analysis

Prism 5.0 software (GraphPad, San Diego, Calif., USA) was used for statistical analysis. The Student's t-test and one-way ANOVA with Bonferroni correction were used to determine statistical significance. Statistical significance was defined as P<0.05.

### Example 10

### The Immunosuppression is Antigen-Specific

The experiments as described herein were also performed with ovalbumin as a control antigen. They showed that the antigen response against ovalbumin is specifically down-regulated after immunization with sia-2,3 and 2,6-conjugated ovalbumin (sia-OVA) and is not due to general suppression of the immune system.

For that purpose, dendritic cells were loaded with sia-OVA and subsequently challenged in the presence of specific CD4+ T cells with either OVA or with a myelin oligodendrocyte glycoprotein peptide (MOG 35-55).

The thus-loaded dendritic cells were then co-cultured with naive MOG-specific CD4+ T cells (called 2D2) in the presence of MOG 35-55 or with naive OVA-specific CD4+ T cells (called OT-II) in the presence of ovalbumin (OVA).

Subsequently, it was determined whether sia-OVA-loaded dendritic cells were able to induce tolerogenic T cells in an antigen-specific manner. This was done by determining the percentage of Foxp3+ CD4+ T cells by flow cytometry.

It was found that dendritic cells loaded with sia-OVA and subsequently challenged with OVA, induced about 10% of tolerogenic cells in a population of OT-II cells, whereas dendritic cells loaded with sia-OVA and subsequently challenged with MOG 35-55 did not induce tolerogenic cells above background levels in a population of 2D2 cells (FIG. 1).

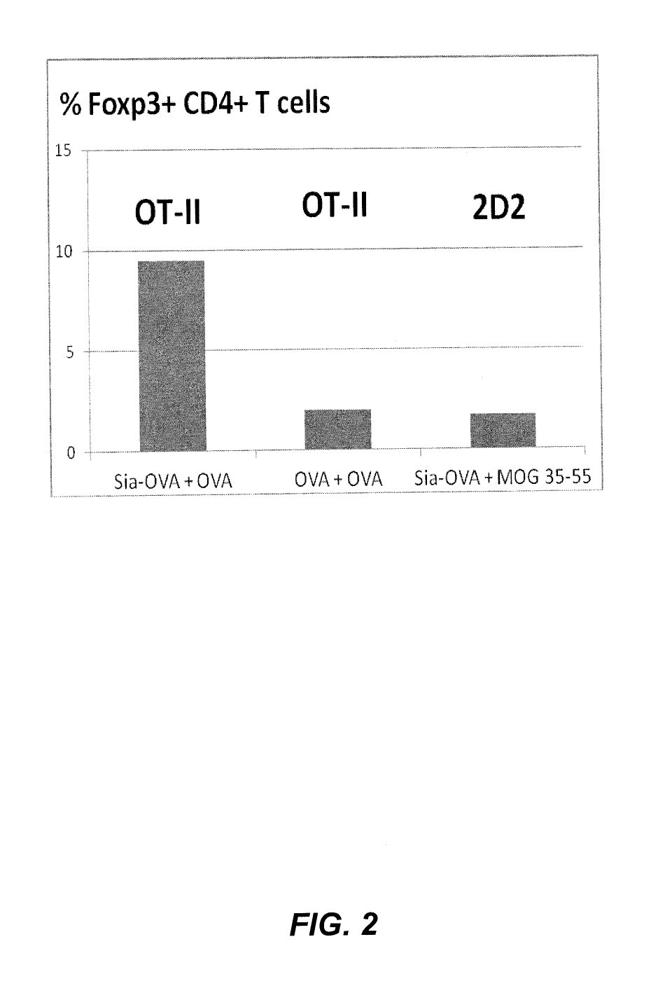

The appropriate negative control experiment with OVA-loaded dendritic cells challenged with OVA did not induce tolerogenic cells above background levels in a population of OT-II cells (FIG. 2).

It may be concluded from these experiments that loading dendritic cells with sia-OVA induced immune tolerance against OVA and that loading dendritic cells with sia-OVA does not induce tolerance against an unrelated antigen (MOG 35-55).

In other terms, this shows that a sia α-2,6-conjugated antigen may be used for suppressing an immune response specifically directed against the antigen in a patient in need of such a treatment.

### REFERENCES

1. Correale J., and S. N. Tenembaum. Myelin basic protein and myelin oligodendrocyte glycoprotein T-cell repertoire in childhood and juvenile multiple sclerosis. Mult. Scler. 2006; 12:412-420.
2. Maier J., C. Kincaid, A. Pagenstecher, and I. L. Campbell. Regulation of signal transducer and activator of transcription and suppressor of cytokine-signaling gene expression in the brain of mice with astrocyte-targeted production of interleukin-12 or experimental autoimmune encephalomyelitis. Am. J. Pathol. 2002; 160:271-288.
3. Miljkovic D., M. Momcilovic, I. Stojanovic, S. Stosic-Grujicic, Z. Ramic, and M.

Mostarica-Stojkovic. Astrocytes stimulate interleukin-17 and interferon-gamma production in vitro. J. Neurosci. Res. 2007; 85:3598-3606.
4. Critchfield J. M., M. K. Racke, J. C. Zuniga-Pflucker, B. Cannella, C. S. Raine, J. Goverman, and M. J. Lenardo. T-cell deletion in high antigen dose therapy of autoimmune encephalomyelitis. Science 1994; 263:1139-1143.
5. Whitacre C. C., I. E. Gienapp, C. G. Orosz, and D. M. Bitar. Oral tolerance in experimental autoimmune encephalomyelitis. III. Evidence for clonal anergy. J. Immunol. 1991; 147:2155-2163.
6. Zhang G. X., H. Xu, M. Kishi, D. Calida, and A. Rostami. The role of IL-12 in the induction of intravenous tolerance in experimental autoimmune encephalomyelitis. J. Immunol. 2002; 168:2501-2507.
7. Jiang et al., Eur. J. Immunol. (2009) 39:789-799.
8. Chen Y., V. K. Kuchroo, J. Inobe, D. A. Hafler, and H. L. Weiner. Regulatory T-cell clones induced by oral tolerance: suppression of autoimmune encephalomyelitis. Science 1994; 265:1237-1240.
9. Miller A., O. Lider, A. B. Roberts, M. B. Sporn, and H. L. Weiner. Suppressor T cells generated by oral tolerization to myelin basic protein suppress both in vitro and in vivo immune responses by the release of transforming growth factor beta after antigen-specific triggering. Proc. Natl. Acad. Sci. U.S.A. 1992; 89:421-425.
10. Weishaupt A., S. Jander, W. Bruck, T. Kuhlmann, M. Stienekemeier, T. Hartung, K. V. Toyka, G. Stoll, and R. Gold. Molecular mechanisms of high-dose antigen therapy in experimental autoimmune encephalomyelitis: rapid induction of Th1-type cytokines and inducible nitric oxide synthase. J. Immunol. 2000; 165:7157-7163.
11. Bettelli E., M. Dastrange, and M. Oukka. Foxp3 interacts with nuclear factor of activated T cells and NF-kappa B to repress cytokine gene expression and effector functions of T helper cells. Proc. Natl. Acad. Sci. U.S.A. 2005; 102:5138-5143.
12. Turley et al., Results Probl. Cell Differ. 2010; 51:217-235.
13. Sharpe A. H., and G. J. Freeman. The B7-CD28 superfamily. Nat. Rev. Immunol. 2002; 2:116-126.
14. Karandikar N. J., C. L. Vanderlugt, J. A. Bluestone, and S. D. Miller. Targeting the B7/CD28: CTLA-4 costimulatory system in CNS autoimmune disease. J. Neuroimmunol. 1998; 89:10-18.
15. Zhang X., R. Hupperts, and M. De Baets. Monoclonal antibody therapy in experimental allergic encephalomyelitis and multiple sclerosis. Immunol. Res. 2003; 28:61-78.
16. Bar-Or et al., Arch. Neurol. (2007) 64(10):1407-1415. Epub 2007 Aug. 13.
17. Jiang Z. et al., Eur. J. Immunol. 2009 March; 39(3):789-99. doi: 10.1002/eji.200838427.
18. Nylander A. and D. A. Hafler. Multiple sclerosis. J. Clin. Invest. 122:1180-1188 (2012).
19. Varki A., R. Kannagi, and B. P. Toole. Glycosylation Changes in Cancer (2009).
20. Crocker P. R., S. J. McMillan, and H. E. Richards. CD33-related siglecs as potential modulators of inflammatory responses. Ann. N.Y. Acad. Sci. 1253:102-111 (2012).

1. A method of treating a subject suffering from multiple sclerosis, the method comprising administrating a sialylated antigen to a subject in need of such a treatment, wherein the antigen is a myelin antigen and wherein the sialylated antigen comprises the sialylated oligosaccharide Neu5Ac(alpha)2-6Gal(beta)1-4GlcNAc.

2. The method according to claim 1, wherein the antigen comprises a peptide, a protein, a glycoprotein, or a lipid.

3. The method according to claim 2, wherein the peptide is an oligopeptide.

4. The method according to claim 1, wherein the antigen is a myelin oligodendrocyte glycoprotein antigen.

5. The method according to claim 1, wherein the antigen comprises SEQ ID NO: 1.

6. The method according to claim 2, wherein the antigen is a myelin oligodendrocyte glycoprotein antigen.

7. The method according to claim 3, wherein the antigen is a myelin oligodendrocyte glycoprotein antigen

8. The method according to claim 2, wherein the antigen comprises SEQ ID NO: 1.

9. The method according to claim 3, wherein the antigen comprises SEQ ID NO: 1.

10. The method according to claim 4, wherein the antigen comprises SEQ ID NO: 1.

11. A method of administering a composition to a subject suffering from multiple sclerosis, the method comprising:
administering a composition comprising a sialylated antigen to the subject, wherein the antigen is a myelin antigen and wherein the sialylated antigen comprises the sialylated oligosaccharide Neu5Ac(alpha)2-6Gal(beta)1-4GlcNAc.

12. The method according to claim 11, wherein the antigen comprises a peptide, a protein, a glycoprotein, or a lipid.

13. The method according to claim 12, wherein the peptide is an oligopeptide.

14. The method according to claim 11, wherein the antigen is a myelin oligodendrocyte glycoprotein antigen.

15. The method according to claim 11, wherein the antigen comprises SEQ ID NO: 1.

16. The method according to claim 12, wherein the antigen is a myelin oligodendrocyte glycoprotein antigen.

17. The method according to claim 13, wherein the antigen is a myelin oligodendrocyte glycoprotein antigen

18. The method according to claim 12, wherein the antigen comprises SEQ ID NO: 1.

19. The method according to claim 13, wherein the antigen comprises SEQ ID NO: 1.

20. The method according to claim 14, wherein the antigen comprises SEQ ID NO: 1.


SEQUENCE LISTING (2 sequences)

SEQ ID NO: 1 - protein, 21 aa, human
MEVGWYRPPFSRVVHLYRNGK

SEQ ID NO: 2 - protein, 21 aa, mus musculus
MEVGWYRSPFSRVVHLYRNGK